(19) United States
(12) Patent Application Publication
(10) Pub. No.: US 2007/0078497 A1
(43) Pub. Date: Apr. 5, 2007

(54) Remote programming of implantable medical devices

(76) Inventor: John P. Vandanacker, Greenfield, MN (US)

Correspondence Address: MEDTRONIC, INC., 710 MEDTRONIC PARK, MINNEAPOLIS, MN, 55432-9924, US

(21) Appl. No.: 11/242,328

(22) Filed: Oct. 3, 2005

(51) Int. Cl. A61N 1/00

(52) U.S. Cl. 607/59

(57) ABSTRACT

A method for remotely programming a medical device, comprising storing a stratification of a plurality of remotely programmable parameters in a plurality of tiers; storing an authorization level for a user wherein the authorization level corresponds to at least one of the remotely programmable parameter tiers; storing a programmed parameter value entered by the user; verifying that the authorization level of the user corresponds to the remotely programmable parameter tier associated with the stored programmed parameter value; and transferring the stored programmable parameter value to the medical device.

### BACKGROUND

1. Field of the Invention

The present invention relates generally to implantable medical device systems and more particularly to methods for remotely programming an implantable medical device (IMD).

2. Description of the Related Art

One goal of a technology-based health care system that fully integrates the technical and social aspects of patient care and therapy is to connect the client with care providers irrespective of separation distance or location of the participants. While clinicians will continue to treat patients in accordance with accepted medical practice, developments in communications technology are making it ever more possible to provide medical services in a time- and place-independent manner.

Previously, clinical services generally required hospital or office visits. For example, if a physician needs to review the performance parameters of an implantable device in a patient, the patient normally had to go to the clinic. Further, if the medical conditions of a patient with an implantable device warrant continuous monitoring or adjustment of the device, the patient would have to stay in a hospital indefinitely. Such a continued treatment plan poses both economic and social concerns. Under this scenario, as the segment of the population with implanted medical devices increases, many more hospitals/clinics and service personnel will be needed to provide in-hospital or in-clinic service for the patients, thus escalating the cost of healthcare. Additionally the patients will be unduly restricted and inconvenienced by the need to either stay in the hospital or make frequent visits to a clinic.

Yet another condition of the past practice requires that a patient visit a clinical center for occasional retrieval of data from the implanted device to assess the operations of the device, gather patient history for both clinical and research purposes, and adjust operational setting as needed. Such data is acquired by having the patient in a hospital/clinic to download the stored data from the implantable medical device. Depending on the frequency of data collection, this procedure may pose a serious difficulty and inconvenience for patients who live in rural areas or have limited mobility. Similarly, in the event a need arises to upgrade the software of an implantable medical device, the patient will be required to come into the clinic or hospital to have the upgrade installed.

Thus, there is a need to monitor the performance of the implantable devices on a regular, if not a continuous, basis to ensure optimal patient care. Further, there is a need to program an implantable device in response to such monitoring procedures to optimize the monitoring and therapy delivery functions of the implantable device. In the absence of other alternatives, this imposes a great burden on the patient if a hospital or clinic is the only center where the necessary frequent follow up, evaluation and programming of the medical devices could be made. Moreover, even if feasible, the situation would require the establishment of multiple service areas or clinic centers to provide adequate service to the burgeoning number of patients having implanted devices worldwide. Accordingly, a programmer unit would connect to an expert medical center to provide access to expert systems and import the expertise to a local environment. This approach would enable unencumbered access to the implanted device or the patient.

To address these needs, a number of proposals have been made to enable remote programming and monitoring of an implantable medical device (IMD) from a centralized patient management system. Using modern communications technologies, data may be transferred from a centralized computer or server to a remote programmer located in the vicinity of a patient for transferring instructions received from the central location to the IMD. With the inherent advantages of a remote patient management system, potential risks associated with remote IMD programming capabilities include inappropriate programming of an IMD or an adverse response to programming changes occurring when a patient is not under medical supervision.

### BRIEF DESCRIPTION OF THE DRAWINGS

FIG. 1 is an illustration of a medical device system in which various embodiments of the present invention may be practiced.

FIG. 2 illustrates typical components of the IMD shown in FIG. 1.

FIG. 3 is a simplified block diagram of major functional components typically included in the external medical device shown in FIG. 1.

FIG. 4 is a schematic block diagram illustrating functional aspects of a remote patient management system according to one embodiment of the invention.

FIG. 5 is a flow chart summarizing aspects of a remote programming method for use with the system shown in FIG. 5 in accordance with one embodiment of the present invention.

FIG. 6 is a continuation of the flow chart shown in FIG. 5 summarizing steps included in a remote programming method for use in programming programmable parameters included in a lower tier of a parameter stratification scheme.

FIG. 7 is a continuation of the flow chart shown in FIG. 5 summarizing steps included in a remote programming method for programming parameters included in a higher level tier of a parameter stratification scheme.

### DETAILED DESCRIPTION

The following detailed description provides a practical illustration for implementing various embodiments of the invention and is not intended to limit the scope, applicability, or configuration of the invention in any way. The present invention is directed toward providing a remote programming method for use with implantable medical device systems that helps ensure safe, secure programming of a medical device in a remote location. The term “remote” as used herein with regard to programming refers to programming operations being performed when the patient having an IMD being programmed is not in the direct physical presence of a clinician or user performing the programming.

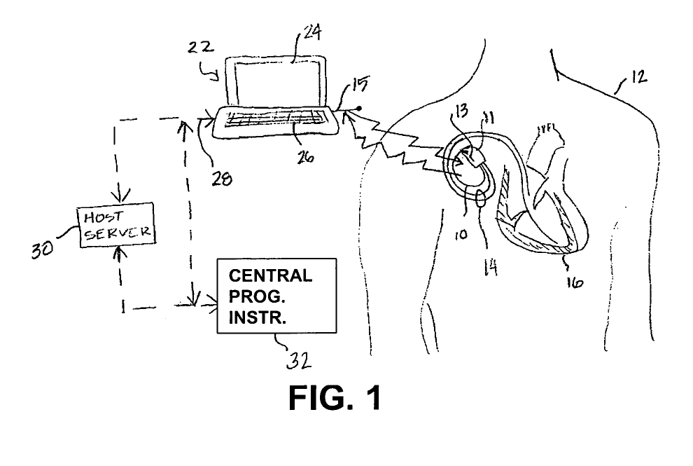

FIG. 1 is an illustration of a medical device system in which various embodiments of the present invention may be practiced. The medical device system includes an IMD 10 and an external medical device (EMD) 22. IMD 10 is shown implanted in the body of a patient 12. The present invention may be implemented for use with a variety of programmable IMDs, including cardiac stimulation devices, cardiac or other physiological monitoring devices, neurostimulators, implantable drug pumps, or the like. For the sake of illustration, IMD 10 is shown here as a cardiac stimulation device coupled to a set of leads 14 used for positioning electrodes and optionally other physiological sensors in operative relation to the patient's heart 16. Leads 14 are coupled to IMD 10 via a connector block 11. Examples of cardiac stimulation or monitoring devices with which the present invention may be employed are disclosed in U.S. Pat. No. 5,545,186 (Olson et al.), U.S. Pat. No. 5,987,352 (Klein et al.), and U.S. Pat. No. 6,438,408 (Mulligan et al.).

IMD 10 is adapted for bidirectional telemetric communication with EMD 22 to allow data stored or being acquired by IMD 10 to be retrieved by EMD 22 during an interrogation or monitoring session. EMD 22 is also used to transfer code, operating parameters, or other instructions to IMD 10. EMD 22 is sometimes referred to as a “home monitor” or “home programmer,” since it is often located in a patient's home such that it is proximate the IMD 10 to enable communication sessions between EMD 22 and IMD 10. EMD 22 may alternatively be located in a hospital room, clinic or other location. Examples of external devices that may be located in a patient's home or in another remote location capable of telemetric communication with an IMD are disclosed in U.S. Pat. No. 6,647,299 (Bourget), U.S. Pat. No. 6,564,104 (Nelson et al.), U.S. Pat. No. 6,561,975 (Pool et al.), U.S. Pat. No. 6,471,645 (Warkentin et al.) and U.S. Pat. No. 6,249,703 (Stanton et al.), all of which patents are incorporated herein by reference in their entirety. EMD 22 may alternatively be embodied as a mobile device that may be worn or carried by the patient.

Programming commands or data are transmitted between an IMD RF telemetry antenna 13 and an external RF telemetry antenna 15 associated with the EMD 22. The external RF telemetry antenna 15 may be contained in a programmer RF head so that it can be located close to the patient's skin overlying the IMD 10. Such programmer RF heads are well known in the art.

See for example U.S. Pat. No. 4,550,370 (Baker), incorporated herein by reference in its entirety. The EMD 22 may be designed to universally program IMDs that employ conventional ferrite core, wire coil, RF telemetry antennas known in the prior art and therefore also have a conventional programmer RF head and associated software for selective use with such IMDs.

Alternatively, the external RF telemetry antenna 15 can be located on the case of the EMD 22, and the EMD 22 can be located some distance away from the patient 12. For example, RF telemetry antenna 15 may be integrated with EMD 22, and EMD 22 may be located a few meters or so away from the patient 12 and utilize long-range telemetry systems. Such long-range telemetry systems allow passive telemetry transmission to occur between IMD 10 and EMD 22 without patient interaction when IMD 10 is within a communication range of EMD 22. Thus, patient 12 may be active, e.g., partaking in normal household activities or exercising during a telemetry transmission. Telemetry systems that do not require the use of a programmer RF head are generally disclosed in U.S. Pat. No. 6,240,317 (Villaseca et al.), U.S. Pat. No. 6,169,925 (Villaseca et al.), and U.S. Pat. No. 6,482,154 (Haubrich et al.), all of which patents are incorporated herein by reference in their entirety.

In an uplink telemetry transmission, the external RF telemetry antenna 15 operates as a telemetry receiver antenna, and the IMD RF telemetry antenna 13 operates as a telemetry transmitter antenna. Conversely, in a downlink telemetry transmission, the external RF telemetry antenna 15 operates as a telemetry transmitter antenna, and the IMD RF telemetry antenna 13 operates as a telemetry receiver antenna. Each RF telemetry antenna is coupled to a transceiver comprising a transmitter and a receiver.

Any of a number of suitable programming and telemetry methodologies known in the art may be employed such as the RF encoded telemetry signal system generally disclosed in U.S. Pat. No. 5,312,453 (Wyborny et al.), incorporated herein by reference in its entirety.

EMD 22 is shown in FIG. 1 to be embodied as a home monitor or home programmer used in conjunction with IMD 10. EMD 22 generally includes a display 24, user interface 26, and a control system typically in the form of one or more microprocessors in addition to the telemetry circuitry described above. However, embodiments of the present invention are not limited to being practiced with an IMD system wherein the external device functions as an associated programmer or home monitor. The present invention may alternatively be practiced with an external medical device system wherein a bedside or portable device performs physiological monitoring or therapy delivery functions. For example, EMD 22 may alternatively be embodied as a bedside monitoring console that may include ECG monitoring, blood pressure monitoring, oxygen saturation monitoring, carbon dioxide monitoring, or other physiological signal monitoring.

Whether EMD 22 is associated with an internal or external medical device system, EMD 22 is provided with a communication link 28 that allows EMD 22 to receive information from and transfer information to a remote patient management system including a centralized programming instrument 32. Centralized programming instrument 32 may be located at a clinical center or other patient management facility and be part of an expert system used for remotely managing IMDs. In one embodiment, centralized programming instrument 32 is a dedicated, microprocessor-based device programmed to execute programming operations and coupled to a communication network. Centralized programming instrument 32 may alternatively be implemented as a web-based programming instrument accessible by an Internet-enabled computer system. Centralized programming instrument 32 is alternatively implemented in programming code on a personal computer. Centralized programming instrument 32 is coupled to a local area network (LAN), wide area network (WAN), telecommunications network, or the like, which allows communication link 28 to be established between central programming instrument 32 and EMD 22. Centralized programming instrument 32 may communicate with EMD 22 via a host server 30, which may be used to control remote programming protocols according to some embodiments of the present invention. Centralized programming instrument 32 may also be accessible from a secondary computer such as a physician's laptop or handheld device via the Internet or other computer network.

It is recognized that a remotely programmable medical device system and associated remote programming methods provided by the present invention may be embodied in a variety of systems, including multiple implantable devices, including various types of EMDs and telemetry systems used for communicating with the IMD(s), and various embodiments of a centralized programming instrument 32 and communication link 28. Centralized programming instrument 32, for example, may be a dedicated instrument or may represent programming functionality implemented in software on an existing computer system or Internet-based web page. Communication link 28 may be established via a modem connection or wireless communication technologies. Additional detailed descriptions of systems for remote management of implantable medical devices in which embodiments of the present invention may be implemented are described in U.S. Pat. No. 6,418,346 (Nelson, et al.), U.S. Pat. No. 6,363,282 (Nichols), U.S. Pat. No. 6,497,655 (Linberg et al.), and U.S. Pat. No. 6,442,433 (Linberg), all of which patents are incorporated herein by reference in their entirety.

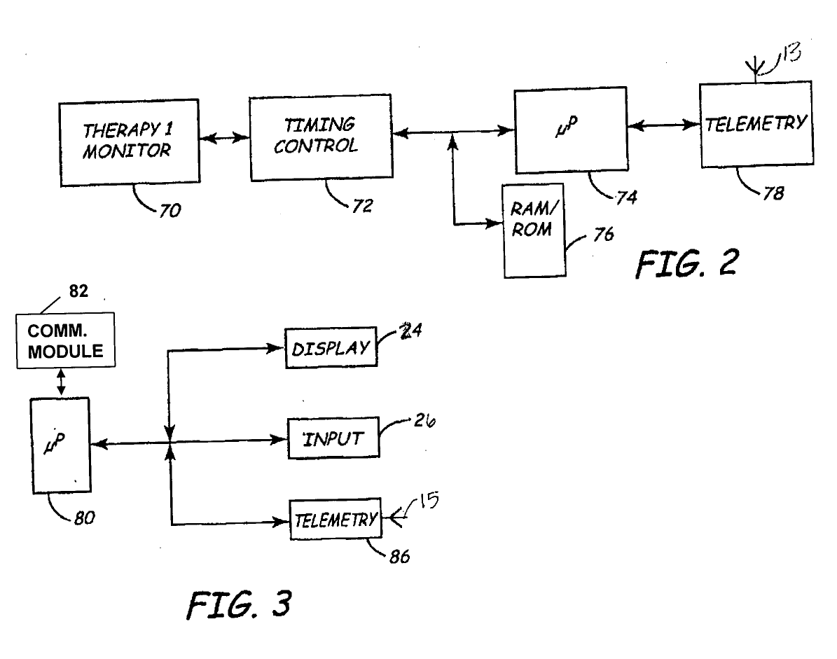

FIG. 2 illustrates typical components of IMD 10 shown in FIG. 1. Major operative structures common to IMD 10 are represented in a generic format. IMD 10 contains timing and control circuitry 72 and an operating system that may employ microprocessor 74 or a digital state machine for timing, sensing and therapy delivery functions in accordance with a programmed operating mode. IMD 10 also contains therapy/monitor 70 which may include sense amplifiers for detecting cardiac signals, patient activity sensors or other physiologic sensors for sensing the need for cardiac output, and pulse generating output circuits for delivering pacing pulses to at least one heart chamber under control of the operating system in a manner known in the art. The operating system includes memory registers or RAM/ROM 76 for storing a variety of programmed-in operating mode and parameter values that are used by the operating system. The memory registers or RAM/ROM 76 may also be used for storing data compiled from sensed cardiac activity and/or relating to device operating history or sensed physiologic parameters for telemetry out on receipt of a retrieval or interrogation instruction. These functions and operations are known in the art, and generally employed to store operating commands and data for controlling device operation and for later retrieval to diagnose device function or patient condition.

Programming commands or data are transmitted between IMD 10 RF telemetry antenna 13 and an external RF telemetry antenna associated with an EMD, as described previously. RF telemetry antenna 13 is coupled to a telemetry transceiver 78. The telemetry transceiver 78 is coupled to control circuitry and registers operated under the control of microcomputer 74. The telemetry transceiver 78 is typically in a low-power state until being “woken-up” for a telemetry session. Telemetry transceiver 78 then operates in a high-power state for sending and receiving data.

Telemetry transceiver 78 may be woken up automatically at programmed intervals of time or based upon detected wake-up signals. One or more timers may be set such that upon expiration of a timer telemetry transceiver 78 wakes up and waits for communication from the EMD. A programmed follow-up interrogation schedule may be implemented using timers for causing the IMD telemetry transceiver 78 to automatically wake up at programmed intervals and wait for an interrogation request from the EMD. In some embodiments, telemetry transceiver 78 is manually woken up with the use of a magnet, tapping or other intervention by the patient or another caregiver.

FIG. 3 is a simplified block diagram of major functional components typically included in an EMD, such as EMD 22 shown in FIG. 1. The external RF telemetry antenna 15 on EMD 22 is coupled to a telemetry transceiver 86, which includes an antenna driver circuit board having a telemetry transmitter and telemetry receiver. The telemetry transmitter and telemetry receiver are coupled to control circuitry and registers operated under the control of microcomputer 80. Telemetry transceiver 86 is used for telemetric communication with IMD 10. EMD 22 further includes a communication module 82, which may be a a hardwired or wireless modem or other communication interface, such as Bluetooth, WiFi, 802.11, or the like, for coupling EMD 22 to a communications network to enable data to be transferred between EMD 22 and the centralized programming instrument.

EMD 22 may be a personal computer type, microprocessor-based device incorporating a central processing unit 80, which may be, for example, an Intel Pentium microprocessor or the like. A system bus interconnects CPU 80 with a storage unit such as a disk drive, storing operational programs and data, and with a graphics circuit and an interface controller module. An external storage unit such as a floppy disk drive or a CD ROM drive may also be coupled to the bus and is accessible via a disk insertion slot within the housing of EMD 22. EMD 22 may include solid-state memory for long-term storage of data.

In order for the physician, patient, or other caregiver or authorized operator to interact with the EMD 22, a keyboard or other user interface 26 coupled to CPU 80 is optionally provided. However, the primary communications mode may be through graphics display screen of the well-known “touch sensitive” type controlled by a graphics circuit. A user of EMD 22 may interact therewith through the use of a stylus, also coupled to a graphics circuit, which is used to point to various locations on screen or display 24 which display menu choices for selection by the user or an alphanumeric keyboard for entering text or numbers and other symbols. Various touch-screen assemblies are known and commercially available. Display 24 and/or the user interface 26 allow a user to enter command signals to initiate transmissions of downlink or uplink telemetry and to initiate and control telemetry sessions once a telemetry link with an implanted device has been established. Other types of user interaction mechanisms and electronics may be implemented such as voice recognition/response systems.

Display screen 24 is also used to display patient related data, menu choices and data entry fields used in entering the data or messages alerting a patient or user to pertinent programming or monitoring conditions. Display screen 24 also displays a variety of screens of telemetered out data or real time data. Display screen 24 may also display uplinked event signals as they are received and thereby serve as a means for enabling the user to timely review IMD operating history and status.

EMD 22 may also include an interface module, which includes a digital circuit, non-isolated analog circuit, and/or isolated analog circuit for coupling peripheral or accessory devices or instruments to EMD 22. The digital circuit enables the interface module to communicate with the interface controller module. For example, EMD 22 may be provided with a strip chart printer or the like coupled to interface controller module so that a hard copy of a patient's ECG, EGM, marker channel of graphics displayed on the display screen can be generated. EMD 22 may be of the type generally disclosed in U.S. Pat. No. 5,345,362 (Winkler), which is incorporated by reference herein in its entirety.

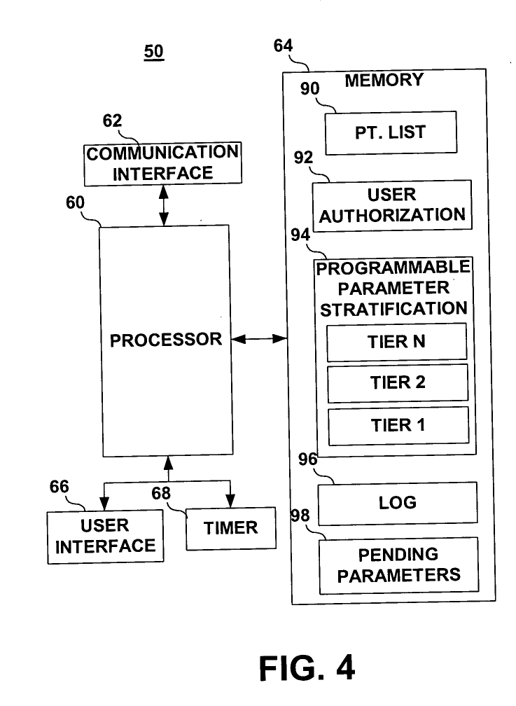

FIG. 4 is a schematic block diagram illustrating functional aspects of a remote patient management system according to one embodiment of the invention. The centralized programming instrument includes a processor 60 for executing programmable code controlling remote programming operations in conjunction with memory 64. A remote programming session will typically be initiated by a physician, nurse, medical technician or other authorized user, generally referred to hereafter as “user,” using the centralized programming instrument. As such a user interface 66 is provided to allow the user to enter log in data, programming data and instructions, and view responses provided by the centralized programming instrument.

The processor 60 determines if a user is authorized to perform remote programming of an IMD based on authorization data stored in memory 64.

Authorization data queried by processor 60 for verifying that a remote programming session initiated by a user is authorized to proceed includes a programmable parameter stratification 94 linked to user authorization levels 92. The programmable parameters included in the IMD are stratified into two or more tiers such that users may be assigned an authorization level that allows or excludes the user from adjusting programmable parameter values corresponding to a particular tier. In one embodiment, the parameter stratification is based on a patient risk level as described in greater detail below. The parameter stratification may be based on nominal assignments of programmable parameters to two or more tiers or a system administrator can enter the parameter stratification using user interface 66. The system administrator also assigns user log in data and corresponding user authorization level stored in user authorization 92. A user authorization level indicates to which parameter tier(s) the user, identified by his/her user log in data, is authorized to make programming changes.

In one embodiment, a first parameter tier includes programmable parameters that are not expected to impact the patient's safety. First tier parameters control “low-risk” IMD functions and generally exclude therapy delivery control parameters. Such parameters may include parameters that control scheduling IMD interrogation sessions or adjusting patient alarm settings. However, first tier programming parameters may also exclude parameters used to control some monitoring functions that generate alert signals. For example, the IMD may be enabled to monitor the fluid status of a heart failure patient for detecting the onset of pulmonary edema. An alert signal generated by the fluid status monitoring function may indicate to a patient or clinician that medical attention, such as an adjustment in medication, is warranted. A user authorized to adjust first tier programmable parameters may not be authorized to adjust parameters that control monitoring functions aimed at prevention or early detection of serious patient conditions in some embodiments.

A second tier of programmable parameters include parameters used for controlling therapy delivery and physiological monitoring of the patient but are not considered to significantly impact patient safety. For example, in a cardiac stimulation device, a second tier parameter may be used to control a pacing lower rate. Limited ranges of programmable settings for second tier parameters may be defined.

A third tier of programmable parameters may be defined which includes parameters controlling device operations that can impact the safety of the patient. For example, therapy delivery control parameters such as defibrillation and cardioversion control parameters may be classified as third tier parameters. Extended ranges of programmable settings of parameters included in the second tier may be included in the third tier. Third tier parameter programming may require additional safety measures such as requiring the patient to be in a clinic or other specified location and/or under supervision (direct or indirect) of medical personnel at the time of programming.

Performing diagnostic testing may be included in a third tier or in a separate higher level tier. For example, running a defibrillation threshold test or performing other electrophysiological studies are associated with an increased risk of an arrhythmia. The risk of a potential arrhythmia associated with an electrophysiological study may require the patient be in a location where an external defibrillator and medical personnel are readily available before remote programming can be performed. Programmable parameter stratification 94 may therefore store associated safety requirements with higher level parameter tiers.

It is recognized that numerous embodiments may exist wherein programmable parameters are stratified in two or more risk-related tiers. The particular parameters and assigned tier levels will depend on the type of device and may be tailored according to individual patient need. A system administrator may determine which personnel are authorized to remotely program each tier level, which may be stored in user authorization 92.

Authorization data used by processor 60 for controlling a remote programming session may further include a patient list 90 linked to user authorization levels 92. The patient list 90 may include patient groupings according to a particular type of IMD, a particular type of diagnosis, having a common primary care physician, a particular risk stratification or other risk-related criteria. A system administrator may determine various patient grouping criteria for which remote programming user authorization status is linked. User authorization data 92 is entered and stored by a system administrator or other authorized personnel to indicate for which patients (or IMDs) a user is authorized to perform remote programming operations.

Memory 64 includes a remote programming log 96 for storing a history of remote programming sessions associated with a given patient or IMD. A user may enter programming notations using user interface 66 for storage in log 96 along with programmed parameter values, date and time information, patient location information, safety requirements, and other relevant data.

Memory 64 further includes allocated space for storing pending programmed parameter values 98 entered by a user but not yet transferred to a targeted EMD. Pending parameter values may be stored for a defined interval of time controlled by the use of a timer 68. Pending parameter values may be canceled if timer 68 expires prior to establishing communication with an EMD and/or verifying successful transfer of parameter values to a targeted IMD.

The remote programming system 50 includes a communication interface 62 for establishing communication with a targeted EMD for transferring user-entered parameter values and receiving parameter verification and/or programming confirmation transmissions from the EMD. The various functional blocks represented in FIG. 4 may be included in centralized programming instrument 32 or distributed across a remote patient management system. For example, data stored in memory 64 may be included in a computer located in a clinic or on a server and accessed by a computer-implemented or web-based centralized programming instrument.

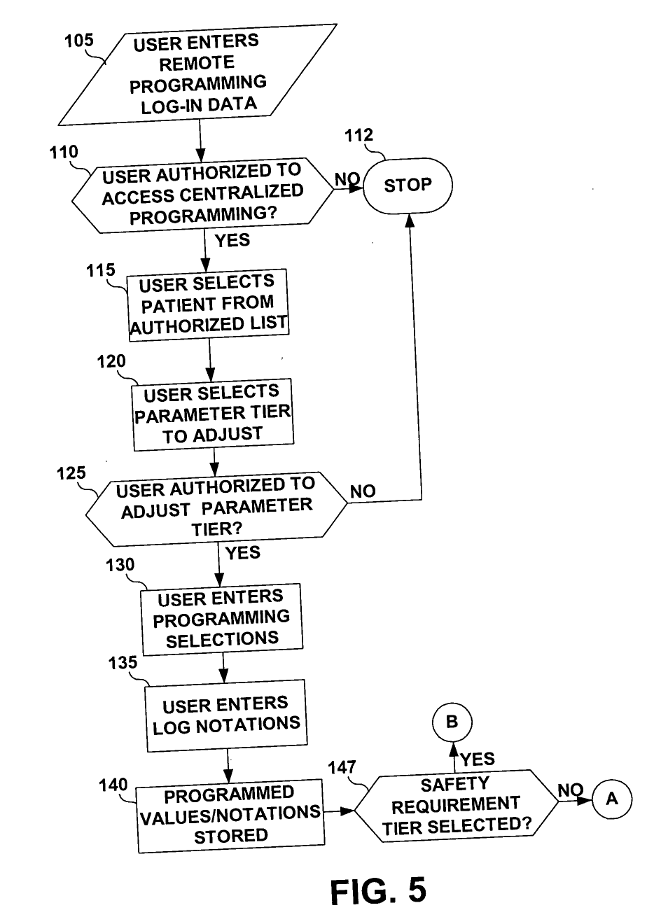

FIGS. 5, 6 and 7 present a flow chart summarizing aspects of a remote programming method according to one embodiment of the invention. A remote programming session is initiated at step 105. At step 105 in FIG. 5, the user enters remote programming log-in data using a user interface to gain access to the centralized programming instrument. In one embodiment, the user enters a secure username and password. In other embodiments, the user gains access to the centralized programming instrument via any implemented secure access protocol such as: a public key/private key protocol; biometric authentication methods which may include a retina scan, fingerprint, voice recognition, or facial image; a user-carried token or swipe card; timed random code or key card; or a predetermined specific series of commands. It is appreciated that numerous protocols may be implemented for allowing secure access of authorized users to the centralized programming instrument.

At step 110, the user's identification is verified as an authorized remote programming user according to user authorization data entered by a system administrator. Some users may be allowed to gain access to a remote patient management system to view data, update patient records, or perform other non-programming functions. If the user is authenticated as an authorized user for performing programming functions, as determined at decision step 110 based on the log-in data provided, the user is presented with a list of patients at step 115. If the user is not authorized to perform remote programming operations, the remote programming method is terminated at step 112.

The user selects a patient at step 115 for which remote programming operations will be performed. The list of patients presented to the user at step 115 includes patients for which the user is authorized to perform remote programming functions. The user may select one or more patients from the presented patient list. In some cases, multiple patients may be selected simultaneously for particular programming operations such as updating remote IMD interrogation scheduling, updating IMD software operating program versions or other operations that may apply to numerous patients simultaneously.

At step 120, the user selects a remote programming parameter tier. Programmable parameters associated with the IMD implanted in the targeted patient(s) are previously stratified into risk-related tiers as described above. As such, the user may select a parameter tier that includes the programmable parameters that the user intends to adjust.

At decision step 125, user authorization for adjusting the selected tier of parameters is verified. If the user is not authorized to adjust programmable parameters included in the selected tier, the remote programming method is terminated at step 112. Alternatively, the user is notified that an attempt to access unauthorized programmable parameters has been made, and the user may be allowed make another selection. In another embodiment, a user may first select any programmable parameter without first selecting a tier. If authorized to make adjustments to that parameter, the user is allowed to proceed with the remote programming session. If the user is not authorized to make adjustments to the selected parameter, the user is given a warning message indicating the unauthorized programming attempt.

At step 130, the user enters programming selections for parameter(s) that he/she is authorized to adjust. The user may enter programming instructions or data that includes software upgrades, operating parameter values, scheduling of remote monitoring sessions, enabling or disabling of IMD functions, or other values, instructions, or data that affect IMD operations. The terms “programmed values” or “programmed parameter values” used herein generally refer to any data, instructions, values or other user-entered information for transfer to the IMD during a remote programming session.

At step 135, the user may enter notations regarding the purpose of the programming session. Such notations may include medical indications for a programmable parameter change or other justifications. Any notations provided by the user explaining the purpose of the programming session will be stored in a remote programming log that allows the history of remote programming operations to be audited.

At step 140, the programmed values entered by the user and any log notations are stored by the centralized programming instrument. Pending programmed values will be stored by the system until a communication link with the appropriate EMD is established.

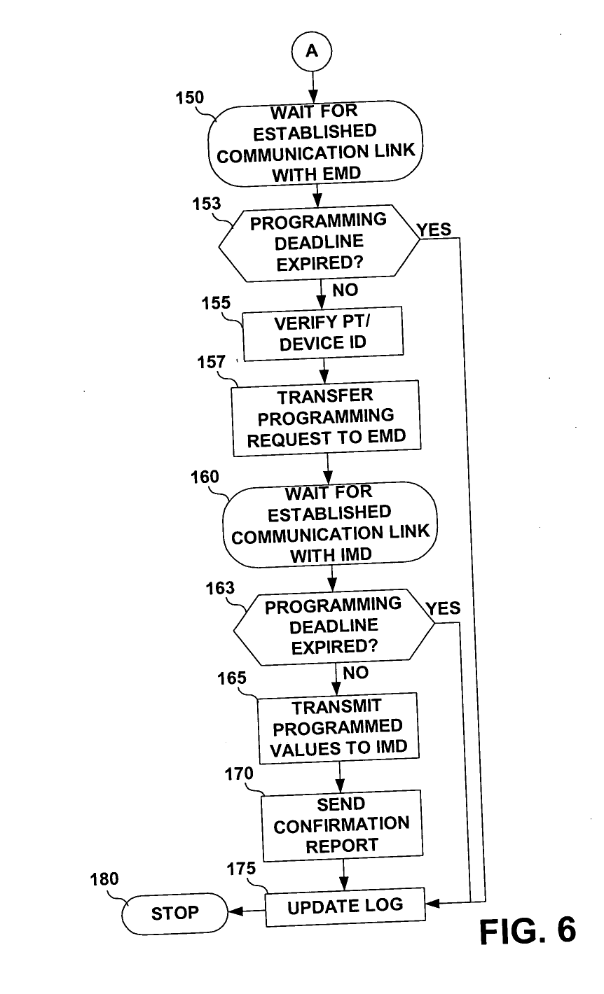

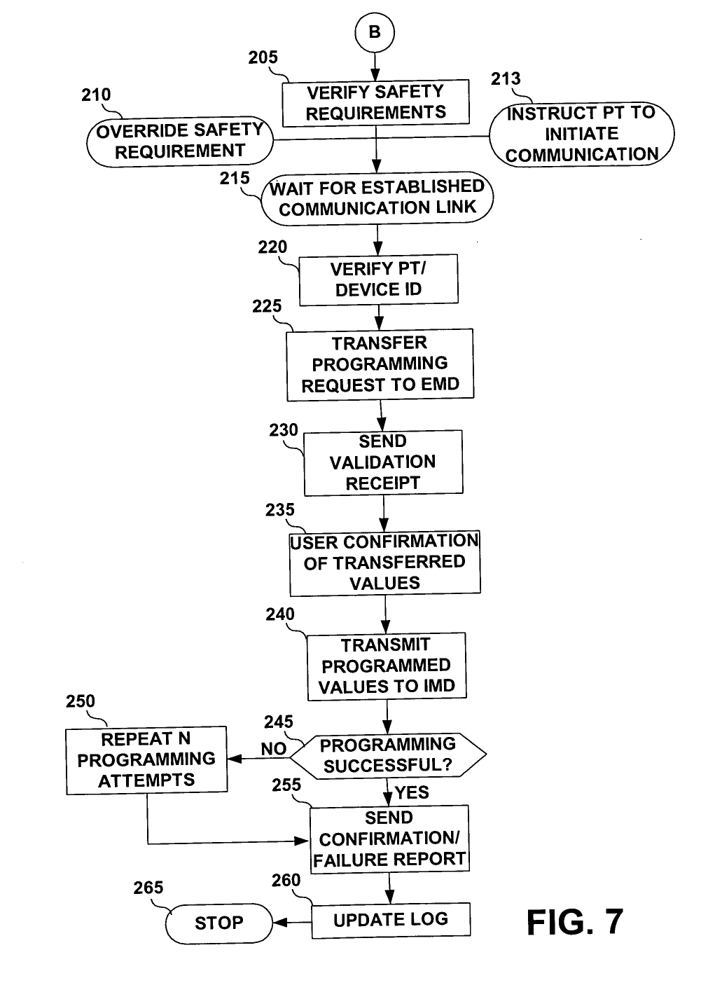

At step 147, a decision is made whether any of the programmed parameter values are associated with a parameter tier that requires additional safety measures to be taken prior to programming the IMD. As described previously, a requirement may be made for the patient to be under supervision or in a medical facility during the remote programming. If the selected parameter is included in a lower level tier that does not require additional safety measures, the remote programming method proceeds to step 150 in FIG. 6 (as indicated by connector “A”). If the selected parameter is included in a higher level tier that does require additional safety measures, the remote programming method proceeds to step 205 in FIG. 7 (as indicated by connector “B”).

FIG. 6 is a continuation of the flow chart shown in FIG. 5 summarizing steps included in a remote programming method for use in programming lower tier programmable parameters. Lower tier programmable parameters are those that do not have an immediate impact on patient safety and therefore do not require additional safety measures at the time of programming. At step 150, the centralized programming instrument waits for a communication link to be established with the appropriate EMD. In some embodiments, the communication link between the centralized programming instrument and the EMD may be available continuously or accessible at any time. In other embodiments, a communication link may be established by the EMD on a scheduled basis.

Once a communication link is established, the centralized programming instrument may verify that a predefined programming deadline has not expired. In one embodiment, the pending programmed values are stored for a maximum amount of time after which the pending programming operation is canceled. If the programming deadline has expired prior to establishing a communication link with the appropriate EMD, the remote programming log is updated at step 175 and the remote programming method is terminated at step 180. The log is updated with a notation regarding the canceled programming operation, the pending programmed values that were canceled, and any notations entered by the user.

If a communication link is established prior to the programming deadline, the centralized programming instrument verifies that the EMD is the appropriate EMD associated with the targeted patient and/or IMD identity for the pending programming operation. Numerous methods for verifying a patient and/or device identification can be used, several of which are described in co-pending U.S. Pat. Appl. Ser. No. 10/871,591, incorporated herein by reference in its entirety.

After verifying that the EMD with which a communication link has been established is the appropriate one for transferring the pending programmed values, the programming request and related data is transferred to the EMD at step 157. At step 160, the EMD waits for a communication link to be established with the IMD. Generally the IMD “wakes up” the IMD telemetry circuitry on a scheduled basis and establishes a communication link with the EMD. In some embodiments, patient interaction is required to wake-up the IMD telemetry.

Once the communication link between the IMD and the EMD is established, the method may once again verify that the programming deadline has not expired at decision step 163. If the deadline has not expired, the pending programmed values are transmitted from the EMD to the IMD. After successfully transferring the programming data, a confirmation report is sent by the EMD to the centralized programming instrument at step 170. Confirmation of successful transmission of the programmed values between the EMD and the IMD can be performed according to telemetry protocols known in the art. Successful transmission may be verified according to protocols for monitoring signal strength, detecting transmission errors, lost data or other subroutines used to verify complete and accurate data transmission. After sending the confirmation report, the remote programming log is updated at step 175, and the remote programming method is terminated at step 180. The log is updated with the programmed parameters, a notation of the confirmation report receipt and any notations entered by the user.

FIG. 7 is a continuation of the flow chart shown in FIG. 5 summarizing steps included in a remote programming method for programming parameters in a higher level tier. Adjustment of parameters in a higher level tier may have immediate impact on patient safety and therefore require one or more additional safety measures at the time of programming. At step 205, the user is prompted by the central programming instrument to verify that any required safety measures have been taken. For example, the user may be required to verify that the patient is at a clinic or under the supervision of appropriately trained medical personnel. The user may be required to verify that emergency equipment is available such as an external defibrillator and a person capable of operating the emergency equipment is readily available should it be necessary.

If the user is unable to verify that the required safety measures are in place, the user may have the option to override the safety requirements in some embodiments as indicated by step 210. In doing so, the user accepts any liability and risk for performing the remote programming operations. After receiving verification or overriding of the required safety measures, the centralized programming instrument waits for a communication link to be established with the appropriate EMD. Meanwhile, the patient is contacted and instructed to establish a communication link between the IMD and the EMD at an appropriate time as indicated by step 213. Since the remote programming operation may be performed at a time that does not correspond to a scheduled interrogation session, intervention by the patient or another caregiver is required to “wake-up” the IMD telemetry in order to establish communication with the EMD. The patient may hold a magnet over the IMD, tap on the IMD, or take other action as instructed to manually “wake up” the IMD telemetry in accordance with the particular telemetry system implemented.

The centralized programming instrument verifies the patient identification, the IMD identification, and/or the EMD identification at step 220 after the communication link to the IMD is established. After verifying that the patient and/or IMD identity, a programming request along with the pending programmed values are transferred from the centralized programming instrument to the EMD at step 225. In one embodiment, the EMD sends a validation receipt back to the centralized programming instrument at step 230. The centralized programming instrument prompts the user to confirm the transferred values at step 235. Any error in the transferred values can be corrected at this time by the user prior to transferring the programmed values to the IMD.

Transmission from the EMD to the IMD of the confirmed but still pending programmed values occurs at step 240. At decision step 245, confirmation of successful transmission and receipt of the programmed values by the IMD is verified. Verification of successful transmission can be performed according to telemetry protocols known in the art. If the transmission is not successful, a predetermined number of transmission attempts may be performed as indicated by step 250. If transmission is still unsuccessful, the stored pending parameters are canceled. At step 255, the EMD generates a report sent to the centralized programming instrument indicating confirmation of a successful transmission of the programmed values or failure of the transmission. In some embodiments, the user may reattempt the programming operation by sending a new programming request with the same or updated programmable parameter values.

After receiving a confirmation or failure report, the remote programming log is updated at step 260 by the centralized programming instrument. The log may be updated with a record of the programmed values, the number of transmission attempts made, the confirmation or failure report, any notations entered by the user, time and date, and location information. The remote programming method is terminated at step 265.

It is appreciated that numerous variations to the method summarized in FIGS. 5, 6, and 7 can be conceived. For example, though not shown in FIG. 7, a programming deadline may be set for programming the higher level tier parameters as was described in conjunction with FIG. 6. If the programming deadline expires prior to successfully programming the IMD, the pending parameter values are canceled. For example, if the patient is unable to be reached in order to receive instructions for establishing a communication link, the pending programmed values may be canceled when the programming deadline expires. Transmission of a validation receipt sent by the EMD back to the centralized programming instrument, as shown in FIG. 7 at step 230, may also be included in the steps shown in FIG. 6 during programming of lower tier parameters. It is recognized that numerous intermediate safety measures may be taken for verifying and validating pending programmed values, patient identity, device identity, timely transmission of programmed values, and successful transmission of programmed values. All of which information may be entered into the remote programming log to allow detailed audits of remote programming operations to be performed.

Thus, a system and method for performing remote programming of a medical device have been presented in the foregoing description with reference to specific embodiments. It is appreciated that various modifications to the referenced embodiments may be made without departing from the scope of the invention as set forth in the following claims.

1. A method for remotely programming a medical device, comprising:
storing a stratification of a plurality of remotely programmable parameters in a plurality of tiers;
storing an authorization level for a user wherein the authorization level corresponds to at least one of the remotely programmable parameter tiers;
storing a programmed parameter value entered by the user;
verifying that the authorization level of the user corresponds to the remotely programmable parameter tier corresponding to the stored programmed parameter value; and
transferring the stored programmable parameter value to the medical device.

2. The method according to claim 1 wherein the stratification of the plurality of remotely programmable parameters in the plurality of tiers corresponds to a patient risk level.

3. The method according to claim 1 wherein storing a programmed parameter value further includes storing a user-entered notation.

4. The method according to claim 1 further including updating a remote programming log after transferring the stored programmable parameter value.

5. The method according to claim 1 further including:
storing a user authorized patient list; and
verifying a patient having the medical device is included in the user authorized patient list.

6. The method according to claim 1 further including verifying a safety requirement for the stored programmed parameter value.

7. The method according to claim 6 wherein verifying the safety requirement includes verifying the patient is under medical supervision.

8. A medical device system, comprising:
an electronic data storage medium for storing a plurality of remotely programmable parameters stratified in a plurality of tiers and for storing an authorization level for a user corresponding to at least one of the plurality of tiers;
a centralized programming instrument operative to store programmed values entered by a user and for verifying the authorization level of the user;
an external medical device adapted to receive the stored programmed values from the centralized programming instrument; and
an implantable medical device adapted to receive the stored programmed values from the external medical device.

9. The medical device system according to claim 8 wherein the centralized programming instrument is implemented on a server as a web-based programming instrument.

10. A remote patient management system, comprising:
means for storing a stratified tier level corresponding to a programmable parameter;
means for verifying authorization of a user for adjusting the programmable parameter; and
means for transmitting a user-entered programmable parameter value to an implantable medical device.

11. The system according to claim 10 further including means for storing a programming notation entered by the user corresponding to the user-entered programmable parameter.

12. The system according to claim 10 further including means for storing a remote programming log.

13. The system according to claim 10 further including means for verifying a safety requirement corresponding to a stored stratified tier level.

14. A computer-readable medium for storing a set of instructions which when implemented in a remote patient management system cause the system to:
store a stratification of a plurality of remotely programmable parameters in a plurality of tiers;
store an authorization level for a user wherein the authorization level corresponds to at least one of the remotely programmable parameter tiers;
store a programmed parameter value entered by the user;
verify that the authorization level of the user corresponds to the remotely programmable parameter tier associated with the stored programmed parameter value; and
transfer the stored programmable parameter value to the medical device.

15. The computer-readable medium according to claim 14 which further causes the system to store a user-entered programming notation.

16. The computer-readable medium according to claim 14 which further causes the system to update a remote programming log after transferring the stored programmable parameter value.

17. The computer-readable medium according to claim 14 which further causes the system to:
store a user authorized patient list; and
verify a patient having the medical device is included in the user authorized patient list.

18. The computer-readable medium according to claim 14 which further causes the system to verify a safety requirement for the stored programmed parameter value.

19. The computer-readable medium according to claim 18 which further causes the system to verify the patient is under medical supervision.
